(19) United States
(12) Patent Application Publication
(10) Pub. No.: US 2020/0001105 A1
(43) Pub. Date: Jan. 2, 2020

(54) Whole Body Health Chamber Device and Method of Use

(71) Applicant: Ken Teegardin, Louisville, CO (US)

(72) Inventor: Ken Teegardin, Louisville, CO (US)

(21) Appl. No.: 16/457,916

(22) Filed: Jun. 28, 2019

Related U.S. Application Data
(60) Provisional application No. 62/690,982, filed on Jun. 28, 2018.

(51) Int. Cl. A61N 5/06

(52) U.S. Cl. CPC A61N 5/06; A61N 2005/0637; A61N 2005/064; A61N 5/02; A61N 2005/0659; A61N 2005/0661; A61N 2005/0662; A61N 2005/0652

(57) ABSTRACT

A whole body health chamber device is disclosed. The whole body health chamber device has a base portion, a lid portion, and a patient treatment area for receiving a patient disposed between the base portion and the lid portion. The health chamber device utilizes a plurality of emitter modules configured to emit radiation into the treatment area. Each of the emitter modules contains a plurality of light emitting diodes configured in an array. Each of the light emitting diodes preferably emits a different wavelength of radiation from the other light emitting diodes. The operation of the light emitting diodes are programmed such that the operation of each diode is controlled independently. The power level of any specific diode may be adjusted through pulse width modulation. The frequency of pulsing may vary for each specific diode. Emitter modules may be grouped together to be controlled in unison.

### PRIORITY

This application claims priority to U.S. Provisional Patent Application Ser. No. 62/690,982, filed on Jun. 28, 2018, the disclosure of which is hereby fully incorporated by reference.

### FIELD OF THE INVENTION

The present invention pertains generally to light emitting diode (LED) therapy devices and more particularly to an LED chamber device and method of treatment of medical conditions with an LED chamber device.

### BACKGROUND OF INVENTION

Therapy devices are known and utilized. Individuals may seek treatment of conditions through the application of radiation. Radiation can be applied to patients in any form, such as through laser therapy. Standard applications are limited though. Most systems require large amounts of energy to apply treatment which can lead to injury to the patient. Other systems provide a once size fits all solution which does not allow a therapist to alter the device adequately for different patients. Other devices which permit a therapist to alter the treatment are time intensive and require that the therapist is constantly present during treatment. What is needed therefore is a radiation treatment device which has low consumption of power which may provide individualized treatment to patients while also utilizing the capability of automation to free up clinical treatment time.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The invention is directed toward a whole body health chamber utilizing emitter modules to provide radiation treatment to a patient. The emitter modules utilize light emitting diodes. In the preferred embodiment the emitter modules have a plurality of light emitting diodes. The pulsing frequency of each of the light emitting diodes are operated independently from the other light emitting diodes. The duty cycle of each light emitting diode is operated independently from the duty cycle of every other light emitting diode.

Still other embodiments of the present invention will become readily apparent to those skilled in this art from the following description wherein there is shown and described the embodiments of this invention, simply by way of illustration of the best modes suited to carry out the invention. As it will be realized, the invention is capable of other different embodiments and its several details are capable of modifications in various obvious aspects all without departing from the scope of the invention. Accordingly, the drawing and descriptions will be regarded as illustrative in nature and not as restrictive.

### BRIEF DESCRIPTION OF THE DRAWINGS

Various exemplary embodiments of this invention will be described in detail, wherein like reference numerals refer to identical or similar components, with reference to the following figures, wherein:

FIG. 1A is a top perspective view of a health chamber device;

FIG. 1B is a side view thereof;

FIG. 1C is a side view thereof;

FIG. 1D is a side view thereof;

FIG. 1E is a side view thereof;

FIG. 2 is a top plan view of a patient using the health chamber device;

FIG. 3 is a schematic view of the components of the health chamber device;

FIG. 4 is a schematic of an emitter module;

FIG. 5 is a schematic of an alternative embodiment of an emitter module;

FIG. 6 is a schematic of an emitter module while in use;

FIG. 7 is a schematic of an emitter module while in use;

FIG. 8 is a schematic of a group of emitter modules being used on a patient;

FIG. 9 is a schematic of the internal components of the health chamber device; and

FIG. 10 is a graph illustrating the optical window of the skin.

### DETAILED DESCRIPTION OF THE PREFERRED EMBODIMENT

The claimed subject matter is now described with reference to the drawings. In the following description, for purposes of explanation, numerous specific details are set forth in order to provide a thorough understanding of the claimed subject matter. It may be evident, however, that the claimed subject matter may be practiced with or without any combination of these specific details, without departing from the spirit and scope of this invention and the claims.

As used in this application, the terms “component”, “module”, “system”, “interface”, or the like are generally intended to refer to a computer-related entity, either hardware, a combination of hardware and software, software, or software in execution. For example, a component may be, but is not limited to being, a process running on a processor, a processor, an object, an executable, a thread of execution, a program, and/or a computer. By way of illustration, both an application running on a controller and the controller can be a component.

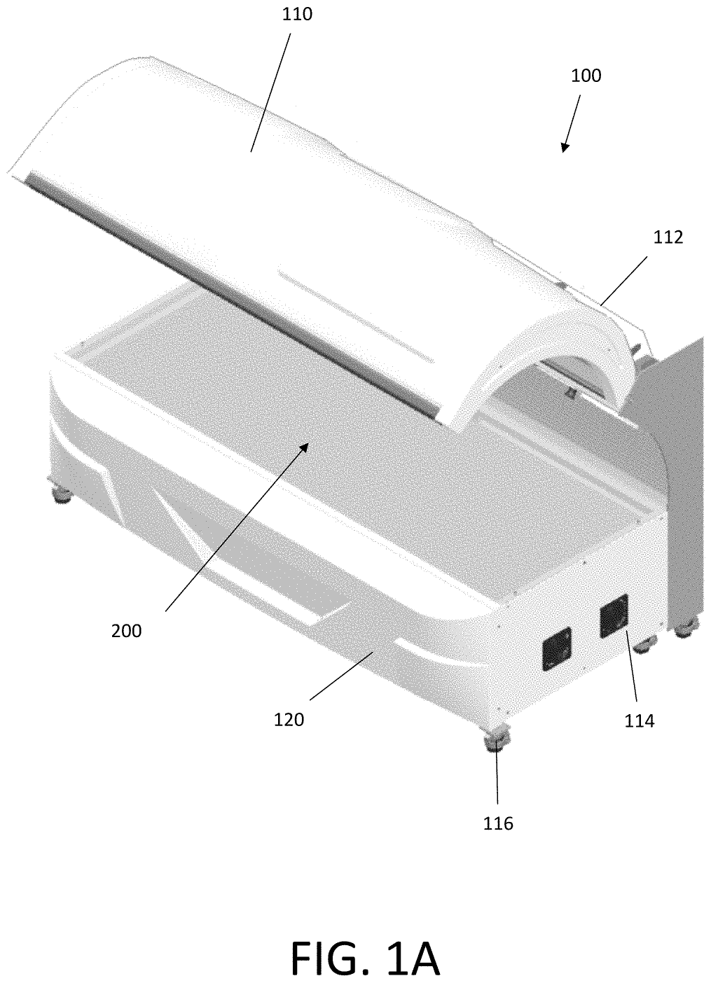

Referring to FIGS. 1A through 1E, the general structure of the health chamber 100 is illustrated. The health chamber 100 is a bed shaped structure which is shaped to receive a patient and provide treatment. The health chamber 100 may be any size and shape and may be situated horizontally or vertically. In the horizontal structure the patient lays down within the treatment area 200. In the vertical structure the patient stands up in the treatment area 200. As shown the health chamber 100 has a base portion 120 and a lid portion 110 which are connected by a hinge 112. The base portion 120 may be any size and shape. In the preferred embodiment the base portion 120 is utilized to house the several internal components of the health chamber 100. The lid portion 110 may be any size and shape. As shown in FIGS. 1B and 1C, the lid portion 110 may be in a raised position to permit the patient to enter the treatment area 200. The lid portion 110 may then be lowered to enclose the patient in the treatment area 200.

The base portion 120 preferably contains one or more fans 114. The fans 114 are utilized to maintain the internal temperature of the base portion 120 and to vent heat created by the internal components of the health chamber 100. The fans 114 may be any size and shape. The fans 114 may vent hot air out of the health chamber 100 or may pull cool air into the health chamber 100. Optionally, there may be one or more fans 114 disposed in the treatment area 200 to provide cooling to the patient. At the bottom of the base portion 120 are a plurality of wheels 116 to permit the health chamber 100 to be maneuvered to any desired location.

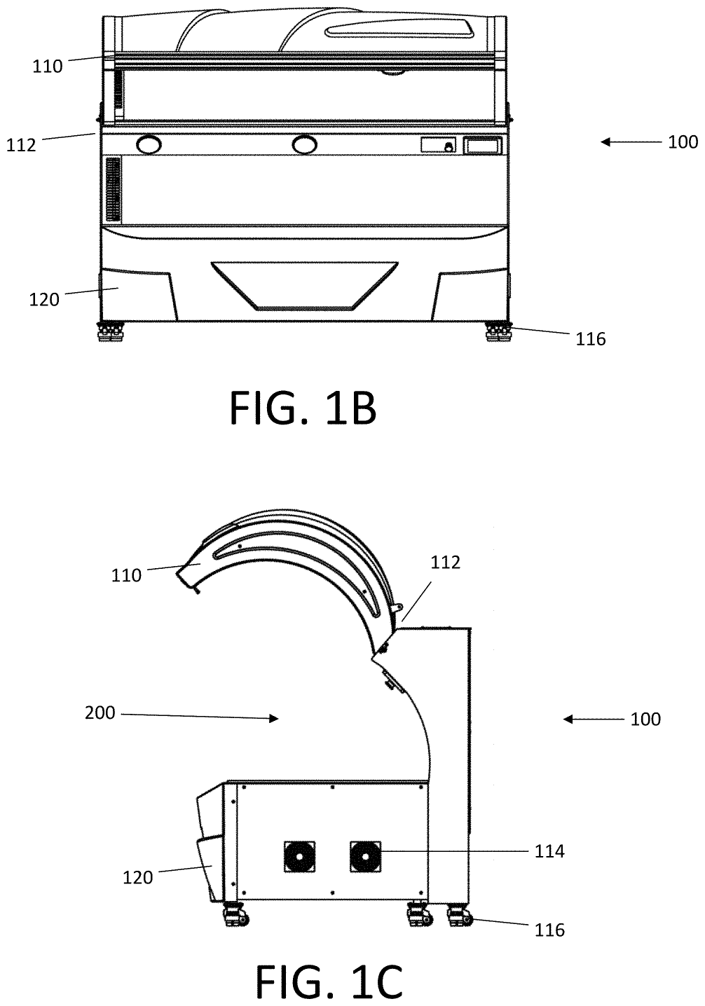

Referring to FIG. 2, a schematic of the health chamber 100 while in use is illustrated. The patient 300 lays prone on the base portion 120. The lid portion 110 is connected to the base portion 120 by the hinge 112. While shown in an open configuration, one knowledgeable in the art would understand the lid portion to be dispose directly above the patient 300. Embedded in the base portion 120 and the lid portion 110 are a plurality of emitter modules 140. The emitter modules 140 may be in any configuration. The emitter modules 140 are disposed facing the patient 300. The emitter modules 140 are configured to emit radiation toward the patient 300. The emitter modules 140 may emit any type of radiation at the patient, including visible light, ultraviolet radiation, microwaves, infrared, x-rays, or any other type of radiation. Each emitter module 140 may be any size and shape. The emitter modules 140 are preferably arranged in a grid format so that all areas of the patient 300 may be treated. The emitter modules 140 may emit radiation directly to the patient 300 or may be passed through one or more filters, refractive or reflective lenses, or reflected to certain portions of the patient 300 by mirrors. The lenses or mirrors may be mounted on motors to turn or angle the lenses or mirrors in any configuration to allow the administrator of the health chamber 100 to focus or direct treatment to specific areas of the patient 300.

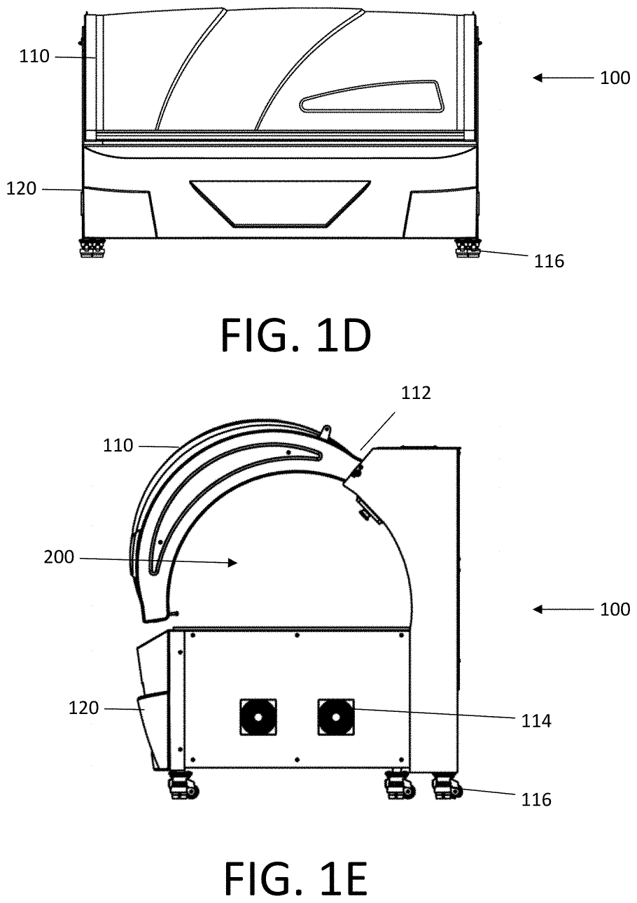

Referring to FIG. 3, the internal components of the health chamber 100 are illustrated. The operation of the health chamber 100 are controlled by the master controller 150. The master controller 150 is a computer processor component and may include memory storage. The master controller 150 stores and executes the functions of the several components of the health device. The health chamber 100 may also have a graphical user interface 160. The user interface 160 is preferably a touch screen display which displays information about the operation of the health chamber 100 and permits an administrator to alter the operations of the health chamber 100. In other embodiments the interface 160 may be knobs, dials, lights, or any other interface for displaying information about the operation of the health chamber 100 or permitting the administrator to alter the operation of the health chamber 100. Connected to the mater controller 150 are the plurality of emitter modules 140. The master controller 150 is also preferably connected to a transceiver 170. The transceiver 170 sends and receives information and instructions, permitting an administrator to run the operation of the health chamber 100 remotely or upload information from the health chamber 100 to the internet 400. To control the operation of the health chamber 100 remotely, the administrator may engage with a client device 500. The client device 500 may utilize special software or a client application to operate the health chamber 100. The client device 500 connects to the health chamber 100 through the internet 400.

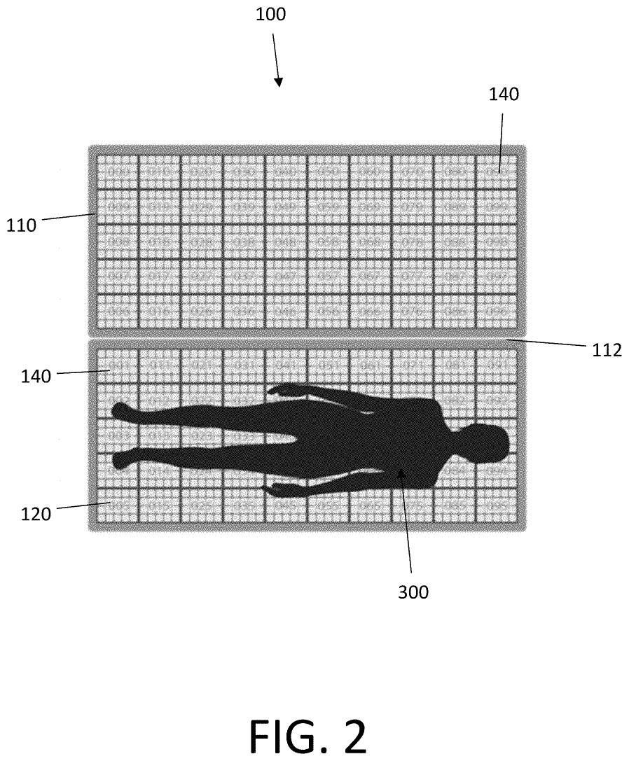

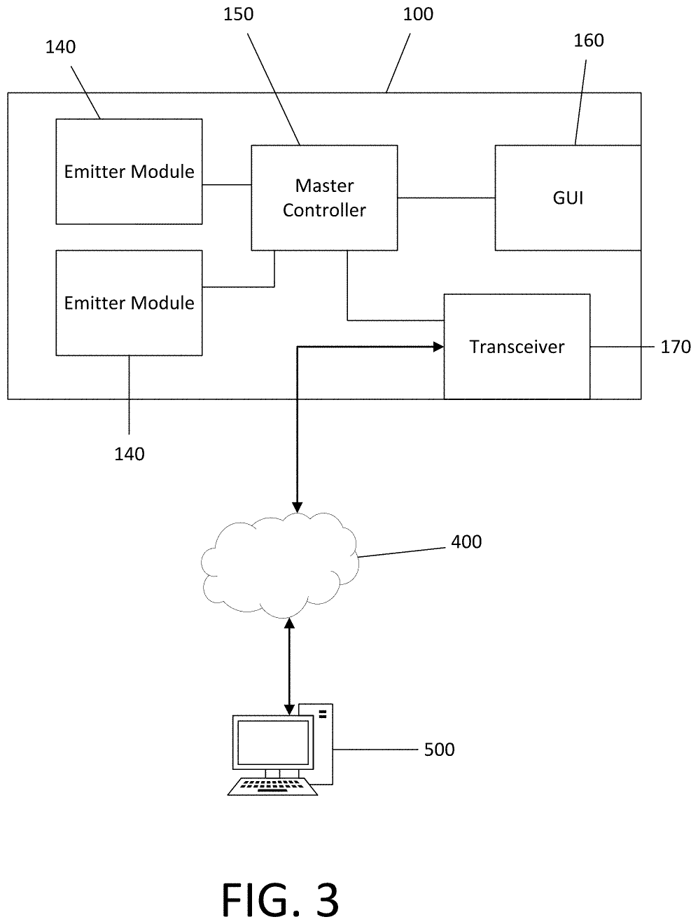

Referring to FIG. 4 the preferred embodiment of the emitter module 140 is illustrated. The emitter module 140 has an array of light emitting diodes (LEDs) 142. There may be any number of LEDs 142. The LEDs 142 may be any type or size. The emitter module 140 may also have a sensor 144. The sensor 144 may be any type of sensor. The sensor 144 may be a temperature sensor, light sensor, motion sensor, or any other type of sensor which provides feedback to the health chamber 100. The LEDs 142 may emanate any wavelength and may emanate the same or difference wavelengths. In the preferred embodiment there is a first LED 142a which emanates a first wavelength, a second LED 142b which emanates a second wavelength, a third LED 142c which emanates a third wavelength, and a fourth LED 142d which emanates a fourth wavelength 142d. Referring to FIG. 5, the emitter module 140 may have more than four LEDs 142. Each LED 142 preferably emits a different wavelength of radiation.

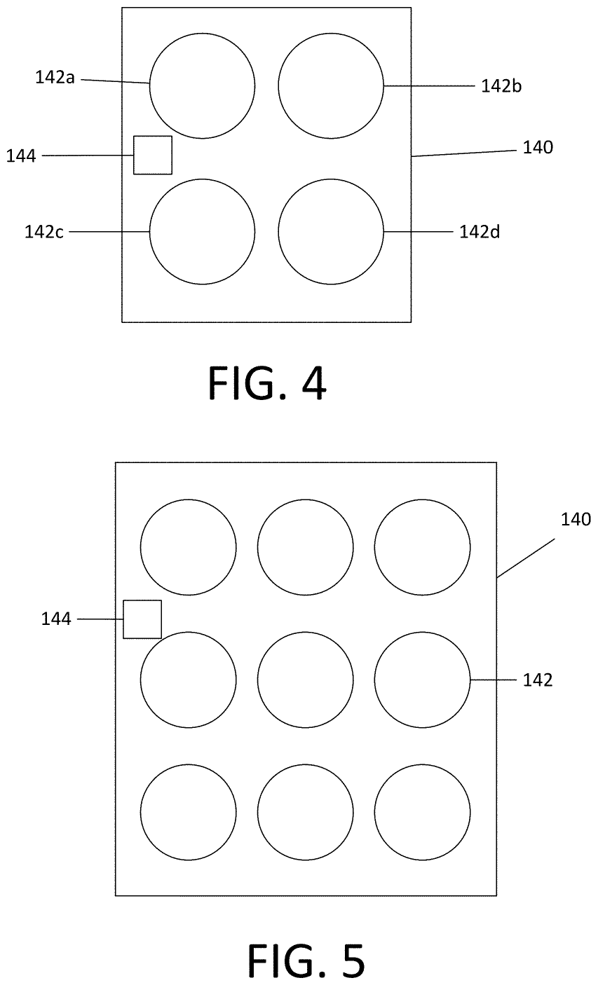

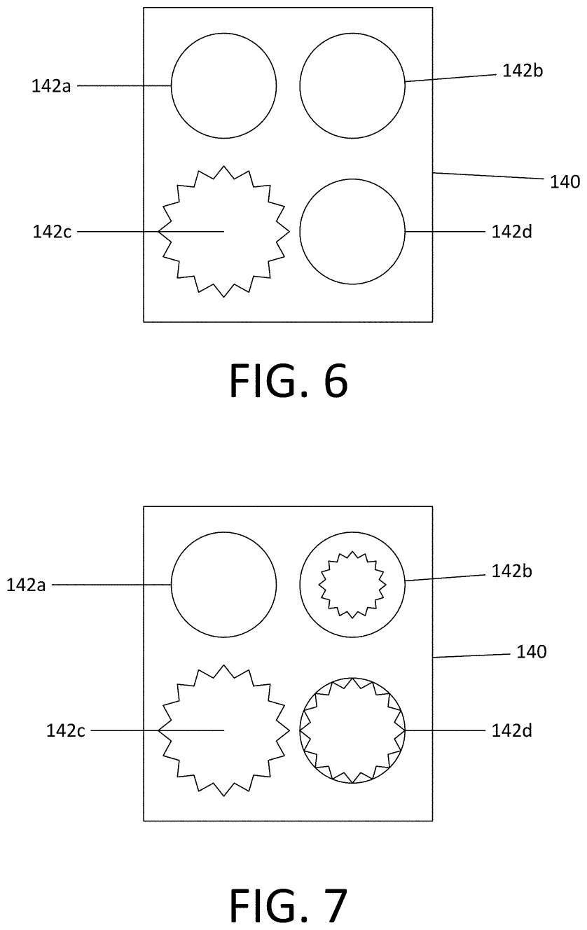

Referring to FIG. 6 and FIG. 7, the operation of the emitter module 140 is illustrated. In the preferred embodiment the first LED 142a has a wavelength of 633 nm, the second LED 142b has a wavelength of 810 nm, the third LED 142c has a wavelength of 850 nm, and the fourth LED has a wavelength of 940 nm. Any of the LEDs 142 may be activated at any time. The emitter module 140 is structured so that the activation and operation of each LED 142 is independent of the other LEDs 142. As shown in FIG. 6, the third LED 142c is activated and emits radiation while the remaining LEDs 142 are inactive. As shown in FIG. 7, the first LED 142a is inactive while the other LEDs 142 are activated. Because the activation of the LEDs 142 are independently controlled the system may alter the power level of each LED 142. As shown in FIG. 7 the power level of the second LED 142b is less than the power level of the fourth LED 142d and the power level of the fourth LED 142d is less than the power level of the third LED 142c.

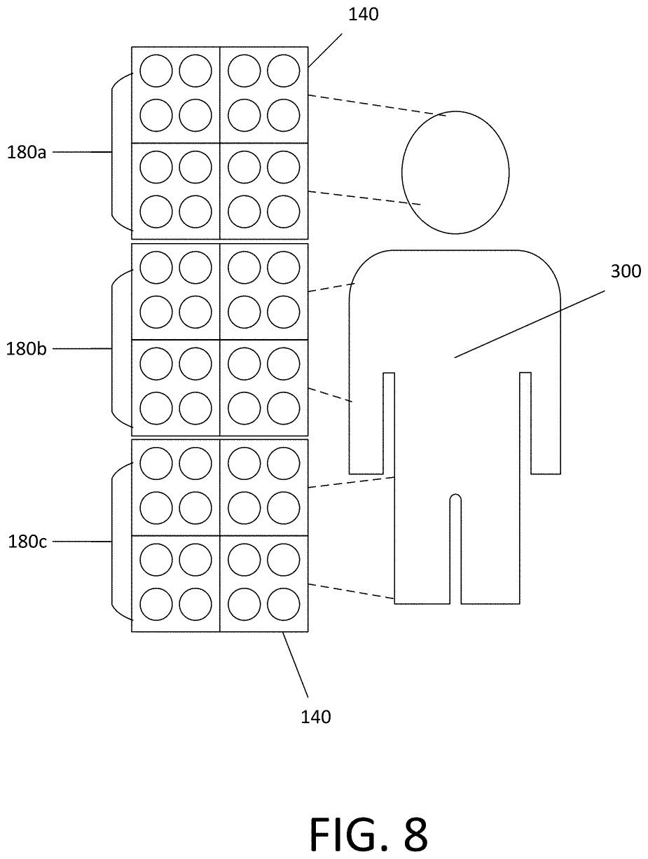

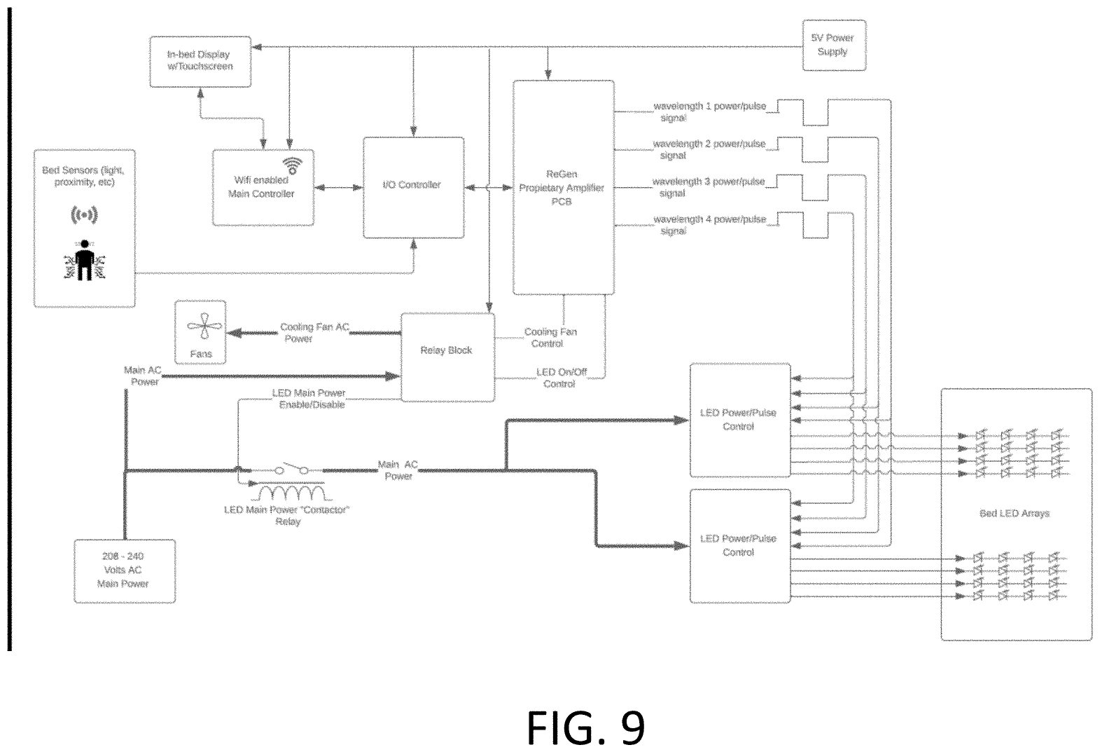

Referring to FIG. 8, the operation of the emitter modules 140 is further illustrated. The emitter modules 140 may be operated together as groups 180. Each group 180 may operate as a unit and operate differently than the other groups 180. The groups 180 may be grouped together to treat different sections of the patient 300. As illustrated, the first group 180a treats the upper portion of the patient 300, the second group 180b treats the middle portion of the patient 300, and the third group 180c treats the lower portion of the patient 300. There may be any number of groups 180. There may be any number of emitter modules 140 in a group 180. The emitter modules in a group 180 may be disposed adjacent to one another as illustrated or may be disposed separately with other emitter modules 140 disposed between them. The group 180 may be static in the number of emitter modules 140 in the group. Alternatively each group 180 may fluctuate such that the emitter modules 140 may enter or leave any group 180 at any time during operation.

The wavelengths utilized in the preferred embodiments may vary from 633 nm, 810 nm, 850 nm, and 940 nm without departing from the scope of the invention. The wavelengths of each LED 142 may be altered+/_20 nm and still work effectively. The system is constructed so that each LED 142 of each emitter module 140 is operated independently from every other LED 142 of each other emitter module 140. The power level of each LED 142 may be independently altered by the master controller 150. The power level of each LED 142 is varied through pulse-width modulation. The system controls the power level of each independent LED preferably by altering the duty cycle of each LED 142. The duty cycle is the ratio of on time to off time for a specific LED 142. The system may also pulse the LED 142 at different frequencies while in use. The pulsing frequency of the power to each LED 142 may be anywhere from 2 Hz to 6000 Hz. Each LED 142 may also be turned on and off in a continuous wave. The operation of the duty cycle for a specific LED 142 is independent of the pulsing for each LED 142 and the change of the duty cycle does not affect the pulsing frequency and the change of the pulsing frequency does not affect the duty cycle.

The power level of each LED 142 may be directly altered by the master controller 150. The master controller 150 may determine that the first LED 142a requires more power than the second LED 142b. The master controller 150 then may boost the voltage to the first LED 142a or decrease the voltage to the second LED 142b. In this way the voltage applied to each LED 142 is independent of the voltage applied to the other LEDs 142.

The system also adjusts the dosage for any specific patient 300. The dosage required to be applied to a patient varies based on the skin color of the patient 300 and the size of the patient 300. The health chamber 100 may track the skin tone of the patient and adjust dosage automatically. The skin tone of the patient may be recorded by a visual sensor or camera which records the skin tone of the patient 300. The recorded skin tone may then be stored and compared to known skin tone colors and preset treatment parameters known for the related skin tone color. The dosage may be optimized for each specific patient 300 to permit the most effective photo-chemical reaction in the treatment are of the patient 300.

In other embodiments the administrator may set the treatment parameters manually for the patient 300 and store the chosen parameters in the health chamber 100 when the patient 300 returns for a later treatment. The health chamber 100 control application can utilize patient feedback to alter the operation of the health chamber device 100. If a patient 300 indicates that a specific treatment was effective then the patient will indicate so. The results may then be entered into the health chamber 100 control application and the information is utilized to compile statistics and demonstrate which programs are most beneficial to the patient 300.

The health chamber 100 stores preset treatment programs which may be given to a patient 300. The preset treatment program preprograms the operations of the LEDs 142. The different wavelengths of the LEDs 142 are utilized to excite different cells in the treatment area of the patient.

The health chamber 100 supports different types of pulsing of the LEDs 142. There may be a basic pulsing where all different wavelengths of the LEDS 142 are pulsed at the same frequency. Alternatively the health chamber 100 may utilize harmonic pulsing where the pulsing occurs at different frequencies but are synchronized. A simple example of this would be pulsing the different wavelengths at 5 Hz, 10 Hz, 15 Hz, and 20 Hz. The health chamber 100 may also utilize light fields where the different LEDs 142 utilize different pulsing frequencies but together they create a synergistic effect. There may be many types of light field pulsing combinations of frequencies. Without limitation, and for an example, one light field may be created by pulsing the four LEDs 142 at 10 Hz, 385 Hz, 3632 Hz and 4625. Hz.

The health chamber 100 allows all these types of pulsing and allow users to use execute them in a fixed format or a sweeping format. In a fixed format the health chamber 100 establishes the pulsing of each LED 142 and does not change it during operation. In sweeping format, the health chamber 100 alters the pulsing through separate frequencies while in use. The health chamber 100 also allows for multi-step programs, Users can also sweep through a set of pulsing frequencies with each step being a different set of pulsing frequencies.

Listed below are examples of classical pulsing and multiple or complex pulsing frequencies. These examples are illustrative of the invention but do not limit the scope of the invention:

Schumann Universal 7.83 Hz (Fixed): This pulsing frequency is described as the universal pulsing frequency of the universe. This frequency can be measure anywhere in space and is thought to be a natural harmonic and all of the wavelengths of the LEDs 142 are pulsed at the same frequency at the same time.

Theta Wave 5-8 Hz (Sweep): These pulsing frequency can be measured in the Brain during sleep. Theta Waves are in a range from 5 Hz to 8 Hz so the health chamber 100 rotates the four different pulsing frequencies across all the wavelengths for quarter of the time (based on the total time of the step) as seen Table 1. Table 1 shows the concept of sweep pulsing and how multiple pulsing frequencies are allocated across multiple wavelengths.

TABLE 1

| Phase | 630 nm | 810 nm | 850 nm | 940 nm |
|---|---|---|---|---|
| 1 | 5 Hz | 6 Hz | 7 Hz | 8 Hz |
| 2 | 6 Hz | 7 Hz | 8 Hz | 5 Hz |
| 3 | 7 Hz | 8 Hz | 5 Hz | 6 Hz |
| 4 | 8 Hz | 5 Hz | 6 Hz | 7 Hz |

Nogier Frequency A 2.28 Hz (Fixed). Resonated the mesoderm and is associated with metabolism, internal organs, nutritional assimilation and a balanced parasympathetic system. This is a simple pulsing frequency where all the wavelengths are pulsed together.

Fi Band-Detox [Fuchtenbusch] (Harmonic Sweep): This pulsing frequency is described as a harmonic light field and is illustrated below in Table 2.

TABLE 2

| Phase | 630 nm | 810 nm | 850 nm | 940 nm |
|---|---|---|---|---|
| 1 | 8 Hz | 442 Hz | 741 Hz | 222 Hz |
| 2 | 222 Hz | 8 Hz | 442 Hz | 741 Hz |
| 3 | 741 Hz | 222 Hz | 8 Hz | 442 Hz |
| 4 | 442 Hz | 741 Hz | 222 Hz | 8 Hz |

Nogier Frequency E Higher Octave 4,672 Hz (Fixed): This pulsing frequency is fixed across all wavelengths and illustrates an example of higher frequency pulsing. The concept is that this pulsing frequency resonates with the spinal cord and peripheral nervous system.

The health chamber 100 allows administrators to set the duty cycle when they create a new pulsing preset. Lower duty cycles can be used for diagnostics purposes. In several publications, they review a process where someone is exposed to lower duty cycle pulsing to see if they have any natural resonances. This information can then be used as input for developing a program. The health chamber may also use a continuous wave adjustable duty cycle.

The health chamber 100 may be preset with programs to operate the health chamber 100. Additionally, an administrator may program a specific program for the operation of the health chamber 100 for any specific patient.

On any remote device, administrators can create unlimited pulsing presets. Each preset defines the pulsing frequency and duty cycle of each the wavelengths of the LEDs 142. Administrators can also turn ON and OFF the option for sweeping through the set of pulsing frequencies. Below is an example of a preset program for utilizing for treatment of a patient 300:

| Step | Preset Name | Step Time |
|---|---|---|
| 1 | Continuous Wave All Wavelengths | 5 minutes |
| 2 | FI Band 01 [Fuchtenbusch] | 3 minutes |
| 3 | FI Band 02 [Fuchtenbusch] | 3 minutes |
| 4 | SOL 5\|528 HZ [Solfeggio] | 2 minutes |
| 5 | Nogier Frequency C Higher Octave\|1,168 Hz | 4 minutes |

This system allows administrators to create any combination of pulsing steps and save the set as a program. The health chamber 100 has an internal library of classical pulsing frequencies. By integrating these pulsing frequencies into the internal library for recall, the health chamber 100 saves administrators the time of doing research and entering the numbers into the system. When building a custom program, administrators simply select for the list of classic pulsing frequencies.

Software programming can be utilized to create safe operation of the health chamber 100. Some users like epileptics are sensitive to flashing red lights which can cause a seizure. The health chamber 100 has a safety feature to prevent this. When this mode is turned ON, the system does not allow the visible red wavelength to pulse at less than 60 Hz. When turned OFF, users can get access to the full range of pulsing options in the visible red spectrum including many of the most popular pulsing frequencies. If many users have access to the pod, turning this mode ON makes the system safer because it stops the system from flashing the visible red light in a range that can trigger a seizure in light-sensitive people. Turning this feature ON is great for unsupervised operation so anyone can use the system and OFF is best if the system has supervised operation where the supervisor will query the user to make sure they are OK with lower red pulsing frequencies. This is an important feature because many of the most documented pulsing frequencies (like the Schumann universal pulsing frequency at 7.83 Hz) are below 60 Hz. This option does not change the pulsing of the invisible IR bands. Since the health chamber 100 has individual pulsing control of each wavelength, users still get the benefit of low frequency IR pulsing when the system is in Epileptic save mode (locking out the red pulsing).

The health chamber device may have an adaptive protocol. When this feature is utilized any user-specific program is adjusted for patient size and skin color following System programming feedback permits administrators or patients 300 to input information about the treatment so that the feedback allows the health chamber 100 to alter the operation for the future.

The health chamber 100 may operate any specific power level in wattage. The health chamber may be low wattage or high wattage.

Any number of LEDs 142 may be utilized. In the preferred embodiment the health chamber 100 has 46,000 LEDs 142.

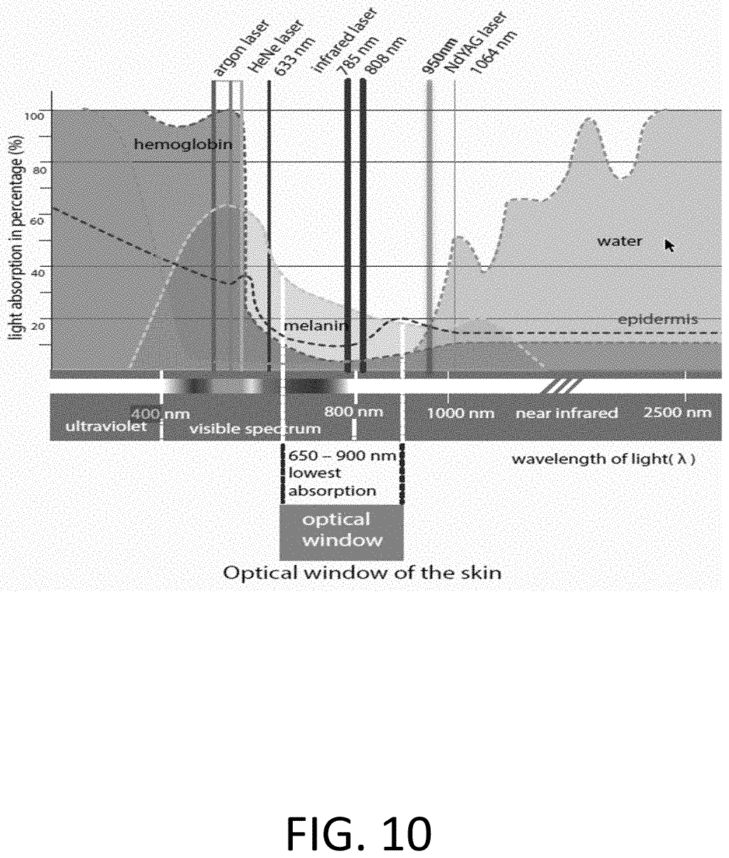

While any specific wavelengths may be utilized for the LEDs 142, the preferred embodiment utilizes four specific wavelengths for nonobvious purposes to take effect of the optical window of the skin of a patient 300. This optical window is illustrated in FIG. 10.

Certain wavelengths can travel through tissue most efficiently and stimulating the release of adenosine triphosphate (ATP) from the mitochondria. There has been a lot of research in this area and some of the research can be summarizes in FIG. 10. In this graph, the optical window (also called the therapeutic window) is the range of wavelengths that provide the best depth of penetration and the desired reaction with the mitochondria. Above 950 nm water starts to become the dominant photonic interaction. Some wavelengths are much better at traveling through tissue and others are absorbed with less benefit. As we approach the lower end of the wavelength graph, we see that more of the energy is absorbed by melanin and then hemoglobin in the blood. As we approach the higher end, we see that water is absorbing much of the energy. Any energy that is absorbed by water cannot be converted into chemical energy. As we see in the graph, higher wavelengths can result in up to 100% of the energy being absorbed by the water in tissue. This is a thermal process that has some benefit by increasing circulation and dilating blood vessels. The design of the health chamber 100 is based on the optical window and the health chamber 100 uses carefully selected wavelengths that provide wavelength diversity while properly balancing the absorption and photochemical response desired.

FIG. 10 illustrates that 650 nm to 900 nm is the preferred wavelength of the LEDs 142 because it minimizes undesired absorption and that wavelengths like 808 nm and 810 nm are a great option for deep penetration applications.

FIG. 10 also illustrates that 940 nm can be a good choice because it is before the rapid slope increase where the energy is mainly absorbed by water in human tissue. By staying at 940 nm and below, wavelength diversity is achieved while avoiding wasting energy that would be absorbed by water in a thermal reaction and not a chemical reaction as desired. At 940 nm, the health chamber 100 give users the maximum flexibility to match the performance of High Intensity Laser Therapy (HILT) class-4 systems and avoids the lower efficiency wavelengths above 940 nm.

Some device designers use 600 nm to 660 nm as the primary wavelength because they want to maximize how much of the energy is absorbed by the melanin close to the surface of the skin. In general, red light systems are best known for treating acne, rashes, wrinkles, and other superficial issues. In fact, most cosmetic devices are based on red LEDs. Even though it is not a good primary wavelength, red can be a good secondary wavelength.

What has been described above includes examples of the claimed subject matter. It is, of course, not possible to describe every conceivable combination of components or methodologies for purposes of describing the claimed subject matter, but one of ordinary skill in the art can recognize that many further combinations and permutations of such matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

The foregoing method descriptions and the process flow diagrams are provided merely as illustrative examples and are not intended to require or imply that the steps of the various embodiments must be performed in the order presented. As will be appreciated by one of skill in the art the order of steps in the foregoing embodiments may be performed in any order. Words such as “thereafter,” “then,” “next,” etc. are not intended to limit the order of the steps; these words are simply used to guide the reader through the description of the methods. Further, any reference to claim elements in the singular, for example, using the articles “a,” “an” or “the” is not to be construed as limiting the element to the singular.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

The hardware used to implement the various illustrative logics, logical blocks, modules, and circuits described in connection with the aspects disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Alternatively, some steps or methods may be performed by circuitry that is specific to a given function.

In one or more exemplary aspects, the functions described may be implemented in hardware, software, firmware, or any combination thereof. If implemented in software, the functions may be stored on or transmitted over as one or more instructions or code on a computer-readable medium. The steps of a method or algorithm disclosed herein may be embodied in a processor-executable software module, which may reside on a tangible, non-transitory computer-readable storage medium. Tangible, non-transitory computer-readable storage media may be any available media that may be accessed by a computer. By way of example, and not limitation, such non-transitory computer-readable media may comprise RAM, ROM, EEPROM, CD-ROM or other optical disk storage, magnetic disk storage or other magnetic storage devices, or any other medium that may be used to store desired program code in the form of instructions or data structures and that may be accessed by a computer. Disk and disc, as used herein, includes compact disc (CD), laser disc, optical disc, digital versatile disc (DVD), floppy disk, and blu-ray disc where disks usually reproduce data magnetically, while discs reproduce data optically with lasers. Combinations of the above should also be included within the scope of non-transitory computer-readable media. Additionally, the operations of a method or algorithm may reside as one or any combination or set of codes and/or instructions on a tangible, non-transitory machine readable medium and/or computer-readable medium, which may be incorporated into a computer program product.

The preceding description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the following claims and the principles and novel features disclosed herein.

1) A health chamber device comprising
a) a base portion;
b) a lid portion;
c) a patient treatment area disposed between said base portion and said lid portion;
d) a mater control unit;
e) a plurality of emitter modules disposed to emit radiation into said patient treatment area;
i) wherein each of said plurality of emitter modules further comprises a plurality of light emitting diodes;
ii) wherein each of said plurality of light emitting diodes are pulsed at a predetermined frequency independent from the frequency of any other light emitting diode; and
iii) wherein each of said plurality of emitter modules has a duty cycle which is independent of a duty cycle of any other light emitting diode.

2) The health chamber device as in claim 1 wherein each of said plurality of light emitting diodes emits a wavelength that is unique and different from a wavelength emitted from the remaining plurality of light emitting diodes.

3) A method for providing treatment to a patient comprising
a) placing a patient in a treatment area of a whole body health chamber;
b) emitting radiation from a first light emitting diode at a first frequency and first duty cycle;
c) emitting radiation from a second light emitting diode at a second frequency and second duty cycle;
d) emitting radiation from a third light emitting diode at a third frequency and third duty cycle; and
e) emitting radiation from a fourth light emitting diode at a fourth frequency and fourth duty cycle;
f) wherein said first light emitting diode, said second light emitting diode, said third light emitting diode, and said fourth light emitting diode emit radiation simultaneously.
